(19) United States
(12) Patent Application Publication
(10) Pub. No.: US 2025/0366757 A1
(43) Pub. Date: Dec. 4, 2025

(54) PLANAR CATHETER WITH STITCHED CONDUCTIVE WIRE

(71) Applicant: Biosense Webster (Israel) Ltd., Yokneam (IL)

(72) Inventors: Pieter Emmelius VAN NIEKERK, Rancho Santa Margarita, CA (US); Babak EBRAHIMI, Lake Forest, CA (US); Debby HIGHSMITH, Laguna Niguel, CA (US); Mohammad ABBAS, Orange, CA (US); Juan RODRIGUEZ SOTO, Irvine, CA (US); Jamie HENRIQUEZ, Coto de Caza, CA (US); Audrey Claire GOO, Irvine, CA (US)

(73) Assignee: Biosense Webster (Israel) Ltd., Yokneam (IL)

(21) Appl. No.: 19/097,672

(22) Filed: Apr. 1, 2025

Related U.S. Application Data
(60) Provisional application No. 63/653,053, filed on May 29, 2024.

(51) Int. Cl. A61B 5/287; A61B 5/00; A61B 5/06

(52) U.S. Cl. CPC A61B 5/287; A61B 5/065; A61B 5/6846; A61B 2560/0468; A61B 2562/046; A61B 2562/166; A61B 2562/227

(57) ABSTRACT

A flexible circuit is herein disclosed. The flexible circuit is intended for insertion into an internal body cavity of a patient. The flexible circuit includes a flexible substrate comprising a bio-compatible material and extending along a substrate plane, a plurality of electrodes disposed on the flexible substrate, and a conductive wire routed along the flexible substrate and connected to one or more of the plurality electrodes by a stitch pattern.

### CROSS REFERENCE TO RELATED APPLICATIONS

This application claims the benefit of priority under 35 U.S.C. § 119 to prior filed U.S. Provisional Patent Application No. 63/653,053, filed May 29, 2024 (Attorney Docket No.: 253757.000494 (BIO6932USPSP1)), the entire contents of which is hereby incorporated by reference as if set forth in full herein.

### FIELD

The present disclosure relates generally to minimally invasive medical devices, and in particular cardiac mapping catheters with flexible probe tips.

### BACKGROUND

Cardiac arrhythmia, such as atrial fibrillation, occurs when regions of cardiac tissue abnormally conduct electric signals to adjacent tissue, thereby disrupting the normal cardiac cycle and causing asynchronous rhythm. Sources of undesired signals can be located in tissue of an atria or a ventricle. Unwanted signals are conducted elsewhere through heart tissue where they can initiate or continue arrhythmia.

Procedures for treating arrhythmia include surgically disrupting the origin of the signals causing the arrhythmia, as well as disrupting the conducting pathway for such signals. More recently, it has been found that by mapping the electrical properties of the endocardium and the heart volume, and selectively ablating cardiac tissue by application of energy, it is possible to cease or modify the propagation of unwanted electrical signals from one portion of the heart to another. The ablation process destroys the unwanted electrical pathways by formation of non-conducting lesions.

In this two-step procedure, which includes mapping followed by ablation, electrical activity at points in the heart is typically sensed and measured by advancing a catheter containing one or more electrical sensors into the heart and acquiring data at multiple points. These data are then utilized to select the target areas at which ablation is to be performed.

For greater mapping resolution, it is desirable for a mapping catheter to conform closely to the target anatomy. For mapping within an atria or a ventricle (for example, an apex of a ventricle), it is desirable for a catheter to collect larger amounts of data signals within shorter time spans. It is also desirable for such a catheter to be capable of allowing sufficient electrode contact with different tissue surfaces, for example, flat, curved, irregular or nonplanar surface tissue, and be collapsible for atraumatic advancement and withdrawal through a patient's vasculature. Existing catheters generally require stiff internal structural members to ensure that a predetermined configuration is maintained. The stiffness is a disadvantage during manipulation in the body organ as it can prevent electrodes from contacting the tissue.

Other catheters can include flexible probe tips designed to overcome this disadvantage. These catheters can include layered components that can be time-consuming, complex, and expensive to manufacture and assemble. Moreover, electrical traces and other components associated therewith can be prone to breakage and/or delamination when in use.

### SUMMARY

There is provided, in accordance with the disclosed technology, a flexible circuit configured for insertion into an internal body cavity of a patient. The flexible circuit includes a flexible substrate, a plurality of electrodes, and a conductive wire. The flexible substrate includes a bio-compatible material and extends along a substrate plane. The plurality of electrodes are disposed on the flexible substrate. The conductive wire is routed along the flexible substrate and connected to one or more of the plurality electrodes by a stitch pattern.

There is further provided, in accordance with the disclosed technology, a medical probe configured for insertion into an internal body cavity of a patient. The medical probe includes a flexible circuit and a plurality of electrodes. The flexible circuit includes a flexible substrate and a conductive wire. The flexible substrate includes a bio-compatible material and extends along a substrate plane having a first side and a second side. The conductive wire is routed along the flexible substrate and connected to the flexible substrate by a stitch pattern. The plurality of electrodes are electrically connected to the conductive wire on only the first side of the substrate plane.

There is further provided, in accordance with the disclosed technology, a medical probe configured for insertion into an internal body cavity of a patient. The medical probe includes an elongated shaft, a probe tip, and a flexible circuit. The elongated shaft extends along a longitudinal axis. The probe tip is connected to the elongated shaft. The flexible circuit extends within the elongated shaft and includes a flexible substrate and conductive wire. The flexible substrate includes a bio-compatible material and extends along a substrate plane having a first side and a second side. The conductive wire is routed along the flexible substrate and is connected to the flexible substrate by a stitch pattern, the conductive wire being electrically connected to the probe tip.

There is further provided, in accordance with the disclosed technology, a medical probe configured for insertion into an internal body cavity of a patient. The medical probe includes a flexible circuit that has a flexible substrate and a conductive wire. The flexible substrate includes a bio-compatible material and extends along a substrate plane having a first side and a second side. The conductive wire is routed along the flexible substrate and is connected to the flexible substrate by a stitch pattern, the conductive wire forming a coil configured to generate a current that is indicative of a position of the coil when the coil is subjected to a magnetic field.

There is further provided, in accordance with the disclosed technology, a medical probe configured for insertion into an internal body cavity of a patient. The medical probe includes a framework and a conductive wire. The framework is substantially planar along a longitudinal axis. The conductive wire (i) forms a coil around a periphery of the framework and (ii) comprises a plurality of spiral loops, each loop extending around a portion of the framework.

There is further provided, in accordance with the disclosed technology, a method of forming a flexible circuit of a medical probe. The method includes the step of stitching, using at least one of a sewn stitch, a woven stitch, or a patched stitch, a conductive wire along a flexible substrate.

There is further provided, in accordance with the disclosed technology, a method of forming a probe tip of a medical probe. The method includes the step of stitching, using at least one of a sewn stitch, a woven stitch, or a patched stitch, a conductive wire along at least a portion of a periphery of the probe tip.

### BRIEF DESCRIPTION OF THE DRAWINGS

FIG. 1 is a schematic pictorial illustration of a medical system including a planar medical probe, in accordance with the disclosed technology;

FIG. 2 is a schematic pictorial illustration of a perspective view of a probe tip, in accordance with the disclosed technology;

FIG. 3 is a schematic pictorial illustration of an exploded perspective view of the probe tip of FIG. 2;

FIG. 4 is a schematic pictorial illustration of a top view of a flexible circuit, in accordance with the disclosed technology;

FIG. 5 is a schematic pictorial illustration of a cross-sectional view of a flexible circuit, in accordance with the disclosed technology;

FIG. 6A is a schematic pictorial illustration of a detail view of a flexible substrate with a conductive wire connecting a plurality of electrodes thereto with a stitch pattern, in accordance with the disclosed technology;

FIG. 6B is a schematic pictorial illustration of a detail view of a flexible substrate with a conductive wire connecting a plurality of electrodes thereto with a stitch pattern, in accordance with the disclosed technology;

FIG. 7A is a schematic pictorial illustration of a detail view of a flexible substrate with a conductive wire with a stitch pattern, in accordance with the disclosed technology;

FIG. 7B is a schematic pictorial illustration of another detail view of a flexible substrate with a conductive wire with a stitch pattern, in accordance with the disclosed technology;

FIG. 7C is a schematic pictorial illustration of another detail view of the flexible substrate and conductive wire, similar to FIG. 7B, in a stretched configuration, in accordance with the disclosed technology;

FIG. 8A is a schematic pictorial illustration of a detail view of an elongated shaft with a long flexible circuit extending therethrough, in accordance with the disclosed technology;

FIG. 8B is a schematic pictorial illustration of a detail view of an elongated shaft with another long flexible circuit extending therethrough, in accordance with the disclosed technology;

FIG. 9A is a schematic pictorial illustration of a perspective view of another probe tip, in accordance with the disclosed technology;

FIG. 9B is a schematic pictorial illustration of a perspective view of another flexible circuit, in accordance with the disclosed technology;

FIG. 9C is a schematic pictorial illustration of a perspective view of a spine framework with a coil wrapped therearound, in accordance with the disclosed technology;

FIG. 10 is a schematic pictorial illustration of a perspective view of another probe tip, in accordance with the disclosed technology;

FIG. 11 is a flow diagram of a method of forming a probe tip, in accordance with the disclosed technology;

FIG. 12 is a flow diagram of a method of stitching a conductive wire to a thermoplastic material, in accordance with the disclosed technology; and

FIG. 13 is a flow diagram of a method of forming a position sensor of a probe tip of a medical probe, in accordance with the disclosed technology.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” or “generally” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 110%. In addition, as used herein, the terms “patient,” “host,” “user,” and “subject” refer to any human or animal subject and are not intended to limit the systems or methods to human use, although use of the subject technology in a human patient represents a preferred embodiment. As well, the term “proximal” indicates a location closer to the operator or physician whereas “distal” indicates a location further away to the operator or physician.

As discussed herein, vasculature of a “patient,” “host,” “user,” and “subject” can be vasculature of a human or any animal. It should be appreciated that an animal can be a variety of any applicable type, including, but not limited thereto, mammal, veterinarian animal, livestock animal or pet type animal, etc. As an example, the animal can be a laboratory animal specifically selected to have certain characteristics similar to a human (e.g., rat, dog, pig, monkey, or the like). It should be appreciated that the subject can be any applicable human patient, for example.

As discussed herein, “operator” can include a doctor, surgeon, technician, scientist, or any other individual or delivery instrumentation associated with delivery of a multi-electrode catheter for the treatment of drug refractory atrial fibrillation to a subject.

The present disclosure is related to systems, methods, uses, and devices for mapping and ablation of cardiac tissue to treat cardiac arrhythmias. Ablative energies are typically provided to cardiac tissue by a tip portion of a catheter which can deliver ablative energy alongside the tissue to be ablated. Some example catheters include three-dimensional structures at the tip portion and are configured to administer ablative energy from various electrodes positioned on the three-dimensional structures. Ablative procedures incorporating such example catheters can be visualized using fluoroscopy.

Ablation of cardiac tissue using application of a thermal technique, such as radio frequency (RF) energy and cryoablation, to correct a malfunctioning heart is a well-known procedure. Typically, to successfully ablate using a thermal technique, cardiac electropotentials need to be measured at various locations of the myocardium. In addition, temperature measurements during ablation provide data enabling the efficacy of the ablation. Typically, for an ablation procedure using a thermal technique, the electropotentials and the temperatures are measured before, during, and after the actual ablation. RF approaches can have risks that can lead to tissue charring, burning, steam pop, phrenic nerve palsy, pulmonary vein stenosis, and esophageal fistula. Cryoablation is an alternative approach to RF ablation that can reduce some thermal risks associated with RF ablation. However maneuvering cryoablation devices and selectively applying cryoablation is generally more challenging compared to RF ablation; therefore, cryoablation is not viable in certain anatomical geometries which may be reached by electrical ablation devices.

The present disclosure can include electrodes configured for RF ablation, cryoablation, and/or irreversible electroporation (IRE). IRE can be referred to throughout this disclosure interchangeably as pulsed electric field (PEF) ablation and pulsed field ablation (PFA). IRE as discussed in this disclosure is a non-thermal cell death technology that can be used for ablation of atrial arrhythmias. To ablate using IRE/PEF, biphasic voltage pulses are applied to disrupt cellular structures of myocardium. The biphasic pulses are non-sinusoidal and can be tuned to target cells based on electrophysiology of the cells. In contrast, to ablate using RF, a sinusoidal voltage waveform is applied to produce heat at the treatment area, indiscriminately heating all cells in the treatment area. IRE therefore has the capability to spare adjacent heat sensitive structures or tissues which would be of benefit in the reduction of possible complications known with ablation or isolation modalities. Additionally, or alternatively, monophasic pulses can be utilized.

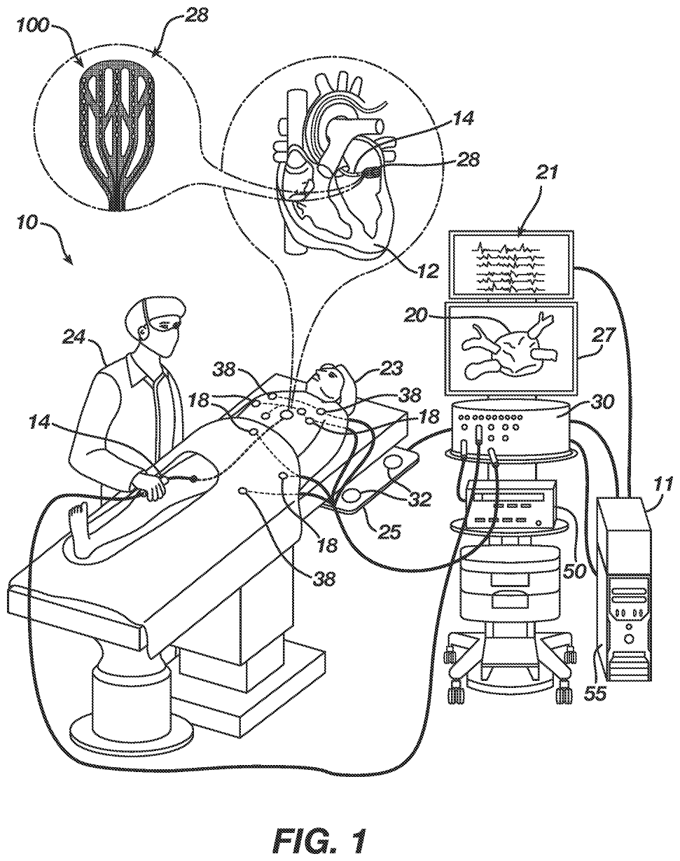

Reference is made to FIG. 1 showing an example catheter-based electrophysiology mapping and ablation system 10. System 10 includes multiple catheters, which are percutaneously inserted by physician 24 through the patient's 23 vascular system into a chamber or vascular structure of a heart 12. Typically, a delivery sheath catheter is inserted into the left or right atrium near a desired location in heart 12. Thereafter, a plurality of catheters can be inserted into the delivery sheath catheter so as to arrive at the desired location. The plurality of catheters may include catheters dedicated for sensing Intracardiac Electrogram (IEGM) signals, catheters dedicated for ablating and/or catheters dedicated for both sensing and ablating. An example catheter/medical probe 14 that is configured for sensing IEGM is illustrated herein. Physician 24 brings a catheter shaft with distal tip of catheter 14 (i.e., multilayered probe tip 100) into contact with the heart wall for sensing a target site in heart 12. For ablation, physician 24 would similarly bring a distal end of an ablation catheter to a target site for ablating.

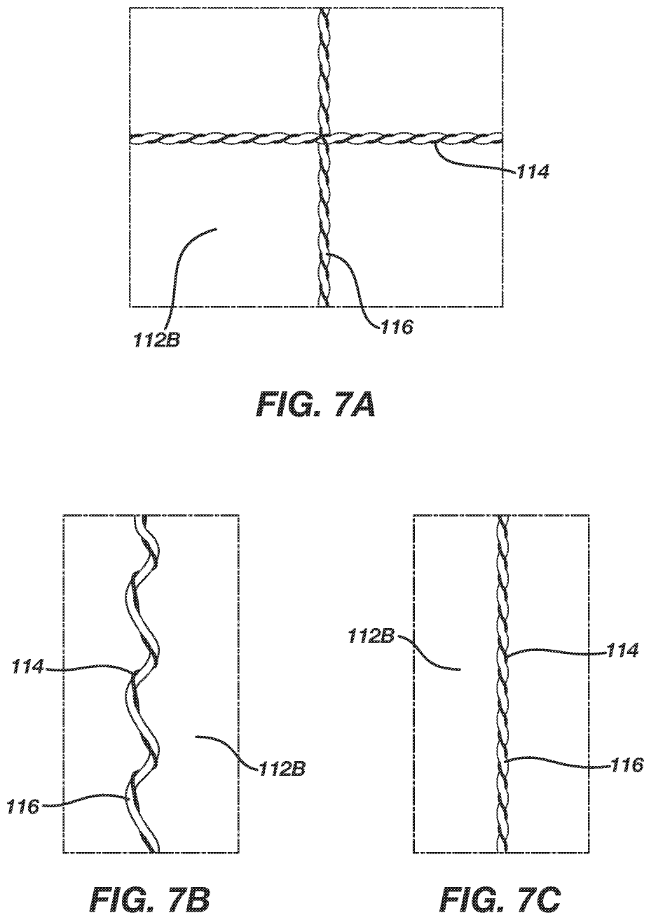

Catheter 14 is an exemplary catheter that includes one and preferably multiple electrodes 112 optionally distributed over probe tip 100 coupled to a catheter shaft and configured to sense the IEGM signals as described in more detail below. Catheter 14 may additionally include a position sensor (see for example the electromagnetic coil 330 in FIGS. 7A-7C) embedded in or near probe tip 100 for tracking position and orientation of probe tip 100. Optionally and preferably, position sensor is a magnetic based position sensor including multiple magnetic coils for sensing three-dimensional (3D) position and orientation (see distal loop 334, first side loop 336a, and second side loop 336b in FIG. 7B).

Magnetic based position sensor may be operated together with a location pad 25 including a plurality of magnetic coils 32 configured to generate magnetic fields in a predefined working volume. Real time position of probe tip 100 of catheter 14 may be tracked based on magnetic fields generated with location pad 25 and sensed by magnetic based position sensor. Details of the magnetic based position sensing technology are described in U.S. Pat. Nos. 5,391,199; 5,443,489; 5,558,091; 6,172,499; 6,239,724; 6,332,089; 6,484,118; 6,618,612; 6,690,963; 6,788,967; 6,892,091, each of which are incorporated herein by reference.

System 10 includes one or more electrode patches 38 positioned for skin contact on patient 23 to establish location reference for location pad 25 as well as impedance-based tracking of electrodes 112. For impedance-based tracking, electrical current is directed toward electrodes 112 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182, each of which are incorporated herein by reference.

A recorder 11 displays electrograms 21 captured with body surface ECG electrodes 18 and intracardiac electrograms (IEGM) captured with electrodes 112 of catheter 14. Recorder 11 may include pacing capability for pacing the heart rhythm and/or may be electrically connected to a standalone pacer.

System 10 may include an ablation energy generator 50 that is adapted to conduct ablative energy to one or more of electrodes 160 at a distal tip of a catheter configured for ablating. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof.

Patient interface unit (PIU) 30 is an interface configured to establish electrical communication between catheters, electrophysiological equipment, power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, processor unit with memory or storage with appropriate operating software loaded therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of multiple catheters within the heart chamber, and (4) displaying on display device 27 sites of interest such as places where ablation energy has been applied. One commercial product embodying elements of the system 10 is available as the CARTO™ 3 System, available from Biosense Webster, Inc., 31 Technology Drive, Suite 200, Irvine, CA 92618.

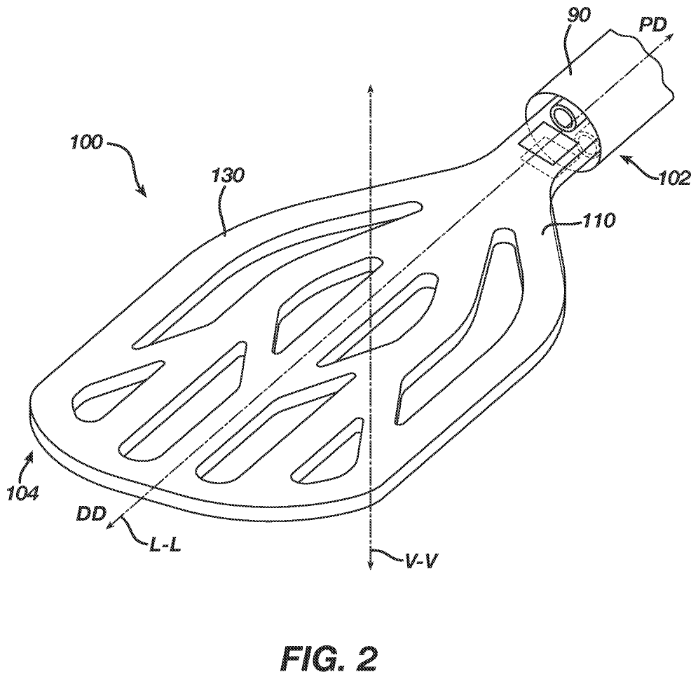

FIG. 2 provides an exemplary probe tip 100, while FIG. 3 shows an exploded view of the probe tip 100, with the components thereof exploded vertically along vertical axis V-V.

The probe tip 100 is configured for insertion into an internal body cavity of a patient and can include a first flexible circuit 110 including a plurality of electrodes 160, each electrode of the plurality of electrodes 160 including a contact surface, as well as a second flexible circuit 150 that includes a plurality of electrodes 160. In some examples, the term “flexible circuit” includes thin-film circuit, flexible printed circuit board, thin film deposition via lithography and etching processes on substrates such as polyimide, copper, LCP, Nitinol substrate, thermoplastic polyurethane (TPU), silicone, thermoset resin, or other polymeric substrates. In some examples, the flexible circuits described herein can be made primarily of polyimide. In other examples, it can be made of any of biocompatible polyimides, glass-reinforced epoxy laminate materials, copper, or graphene, alone or in combination. Other appropriate materials that are in accordance with the present disclosure are discussed in greater detail below. In some examples, the electrodes described herein can include at least one mapping electrode and/or at least one ablation electrode and can be configured to detect electrophysiological signals or transmit ablative energy AC or DC from an energy generator to the tissue according to the various ablation methods previously described e.g., RF, IRE, etc.

The flexible circuit 110 can be disposed in an insulative material 130. The insulative material 130 can be contiguous to the contact surfaces of the electrodes 160 so that only the contact surfaces of at least a portion of the plurality of electrodes 160 are exposed to the ambient environment. As used herein, “ambient environment” refers to the external environment such as the organ in which the probe tip 100 is deployed or in the operating theater prior to being deployed in the biological organ. The insulative material 130 at least partially encapsulates and spaces the different layers of the probe tip 100 (e.g., the flexible circuits 110, 150 and the framework 120, which is discussed in greater detail below) along a vertical axis V-V.

It is noted that not all of the electrodes on the probe tip 100 described herein need be exposed through the insulative material 130 as these non-exposed electrodes can be used to sense far-field signals for noise reduction proximate the tissue contacting electrodes. Similarly, far-field signals including noise or artifacts can be reduced or canceled out for the overall probe tip with a reference electrode that is not in contact with tissues and only with blood.

The probe tip 100 can further include a framework 120 contiguous to the insulative material or in the insulative material. In examples in which the probe tip 100 includes framework 120, the framework 120 can be disposed directly on the flexible circuit 110 with none, or very little, of the insulative material 130 coming between the two. In other examples, as discussed above, the insulative layers 130a, 130b, 130c can space the framework 120 from the flexible circuits 110, 150.

In some examples, the framework 120 is disposed in the insulative material 130 and is substantially planar along a longitudinal axis L-L such that the longitudinal axis L-L is parallel to or coincident with the framework 120. Framework 120 can include a first side 122 and an opposite second side 124 relative to the longitudinal axis L-L. The framework 120 is also generally parallel with a plane defined by the flexible circuit 110 (e.g., plane 113 discussed further below). In the example shown in FIG. 2, a plurality of location sensing loops (not shown) can be provided that can be sandwiched between the flexible circuit 110 and the spine framework 120 and generally parallel thereto. In some examples, the framework 110 is formed from a flexible, resilient material. By way of example, the framework can be formed from a shape-memory alloy such as nickel-titanium, also known as Nitinol, cobalt chromium, stainless steel, and/or other alloys that exhibit pseudo-elastic and/or super-elastic properties.

Stated otherwise, an aspect of the present disclosure provides a probe tip 100 having a planar framework 120 bisecting two flat, heat formed portions 130a, 130b of a flexible insulating mass 130, with at least one flexible circuit 110 (and/or flexible circuit 150) disposed on one side of the framework 120.

Framework 120 can be a component of the probe tip 100 that is separate and distinct from the first flexible circuit 110 and disposed proximate the first flexible circuit 110. In this case, the insulative material 130 can be further disposed between the framework 130 and the second flexible circuit 140. The framework 120 can be formed from a planar or cylindrical stock of material using any suitable method. For example, the framework 130 can be formed by cutting, laser cutting, stamping, etc.

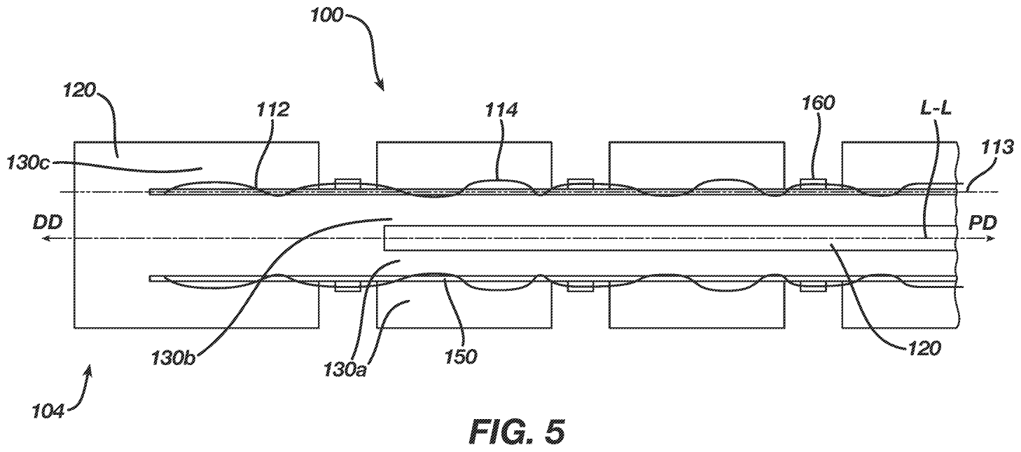

Insulative material 130 can include a first sheet of insulative material 130a, a second sheet of insulative material 130b, and/or a third sheet of insulative material 130c (FIG. 5) fused together proximate the framework 120 into a single, contiguous, generally planar insulative mass 130. This insulative material 130 also serves to enhance the atraumaticity of the probe tip 100 and to protect the subject from sharp edges. The insulative material 130 can include polymer. The insulative material 120 can be heat formed around at least a portion of the first flexible circuit 110, the second flexible circuit 150, and the framework 120. The polymer can include TPU or other heat formed or shaped material which lends itself to said heat forming.

Furthermore, while the insulative material 130 is shown to be flat in these figures, insulative material 120 can be shaped, scalloped, ribbed, ridged, concaved, convexed, or otherwise configured such that the overall profile of insulative material 130 yields physical and/or mechanical properties, such as rigidity and flexion along multiple axes, required by the probe tip 100, mentioned above.

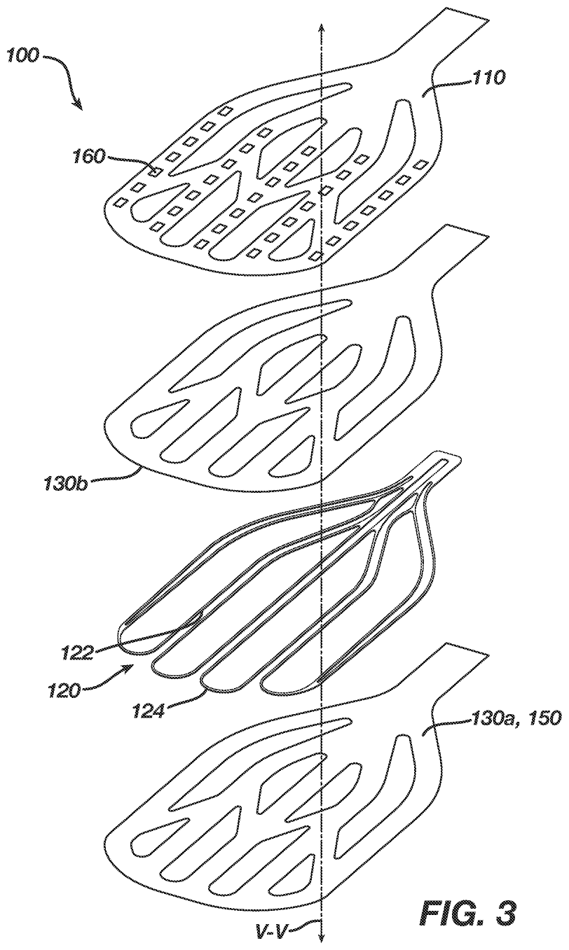

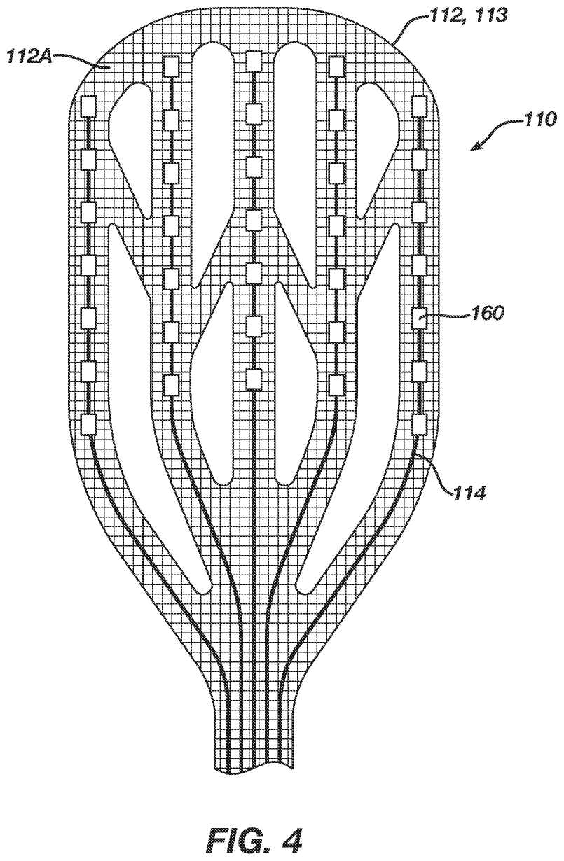

FIG. 4 shows a top view of the flexible circuit 110 that extends along the longitudinal axis L-L, in accordance with the present disclosure. It is noted that the flexible circuit 150 can be configured in a similar manner; therefore, the following description applies to both flexible circuits 110, 150 depicted in the figures. FIG. 5 shows a cross-section taken along the longitudinal axis L-L. It should be noted that while framework 120 is shown in cross-section as rectangular, the framework 120 is not limited such cross-section and any suitable cross-sections can be utilized. With reference now being made to FIGS. 4 and 5, the flexible circuit 110 includes a flexible substrate 112, multiple electrodes 160, and one or more conductive wires 114.

The flexible substrate 112 comprises a bio-compatible material and extends along a substrate plane 113 that bisects the flexible substrate and has a first side and a second side. In some examples, the flexible substrate 112 is formed entirely from or about entirely from the bio-compatible material. As seen in FIG. 4, the bio-compatible material can include a medical grade fabric material 112A. In some examples, the substrate material 112A can be conductive and act as a current drain.

The electrodes 160 are disposed on a surface of the flexible substrate 112. As seen particularly in FIG. 5, the electrodes 160 are disposed on only one side of the plane 113. Put another way, the electrodes 160 are directed only upwardly (relative to the orientation of FIG. 5) so as to face away from the framework 120.

The conductive wires 114 form a part of the electrical interconnections that electrically connect the electrodes 160 with the PIU 30. The conductive wires 114 are routed along the flexible substrate and can be connected to the flexible substrate 112 (which, as discussed above, can be formed from a medical grade fabric material 112A) and/or one or more of the electrodes 160 by a stitch pattern.

By way of example, the wires 114 can be sewn into the fabric material 112A and routed to an electrode 160, which is adhered or connected in any other appropriate manner. At this intersection/connection, the wire 114 can be stripped and welded or soldered to the electrode to form the electrical interconnection therebetween. Alternatively, the wire 114 can be stripped. Then, an electrode 160 is positioned over the wire 114 and riveted immediately over the wire 114 to create mechanical contact. This approach can work without stripping the wire 114, where one or more sharp points in the electrode rivet 160 push through the insulation in the wire 114 and create contact with the wire 114.

By way of example, the conductive wires can be formed from various alloys, such as, but not limited to, copper, Monel, drawn filled tubing (DFT) (e.g., a nickel cobalt alloy (such as MP35N) with a silver core, Nitinol with a platinum core, etc.) Nitinol, and combinations thereof to optimize performance of mechanical, electrical, magnetic and other properties of the wires 114. The wires 114 can be insulated or uninsulated depending on their spacing and the configuration of the substrate 112. In some examples, the wires 114 can be formed from high strength or super-elastic alloys and can be used as a structural element, an electrical element, or both. Alternatively or additionally, the substrate 112 can be sewn/stitched with other conductors, such as twisted pairs of magnetic wire, copper/constantan thermocouple wire, bundled shielded cables, and various combinations/permutations thereof depending on use. As used herein, the term “wire” includes a conductive member of any cross section (e.g., circular, rectangular etc) drawn into a flexible configuration.

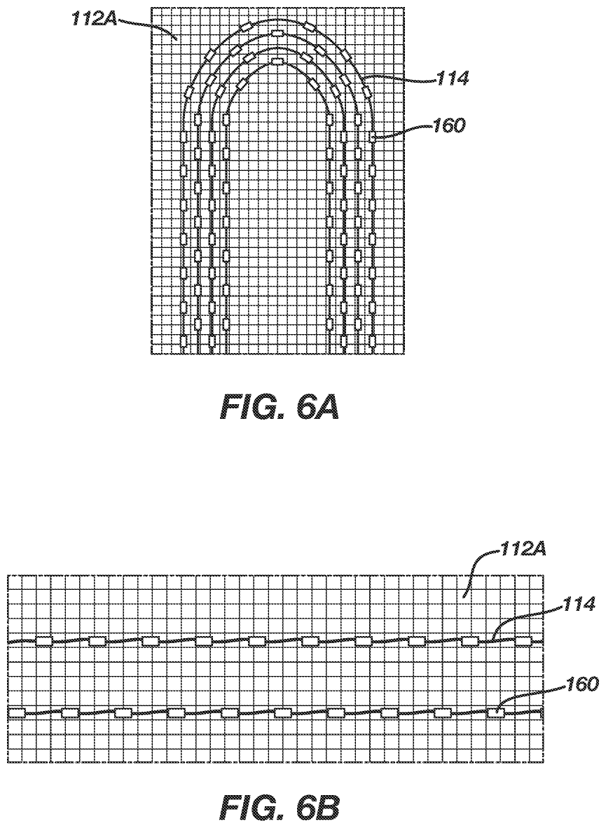

Exemplary stitch patterns are depicted in FIGS. 4-6B. For example, as exemplified in FIG. 4 the stitch pattern can include a patched stitch. As seen in FIG. 5, the stitch pattern can include a sewn stitch where the conductive wire 114 is routed along the flexible substrate 112 in an alternating pattern between the first side and the second side of the substrate plane 113. FIGS. 6A and 6B depict exemplary woven stitch patterns where the conductive wire 114 is woven the fabric material 112A such that the conductive wire 114 does not pass through the plane 113 (and thus can remain on only one side thereof). Moreover, other patterns, such as spiraling and zig-zag stitching patterns (discussed in greater detail below), can be used without departing from the spirit and scope of the present disclosure. As those skilled in the art will appreciate, a combination of these (and other stitches) can be employed at various locations of the probe tip 100.

As shown in FIGS. 4-6B, the substrate 112 can be stitched/sewn with one or more lead conductive wires 114 in various orientations/directions, each terminating at specific locations. Afterwards, conductive rivets can be secured over the wire 114 where it contacts a conductive component of the wire 114. In this example, the rivet can function as an electrode 160.

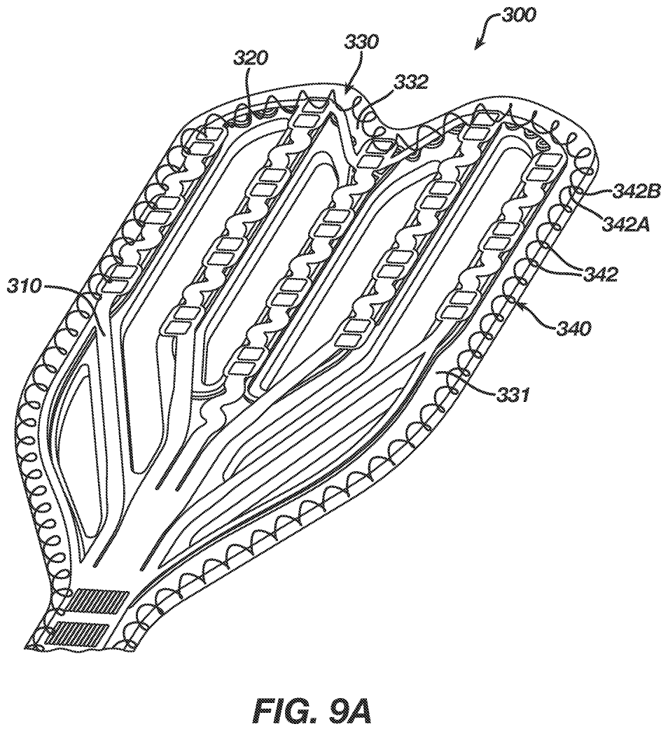

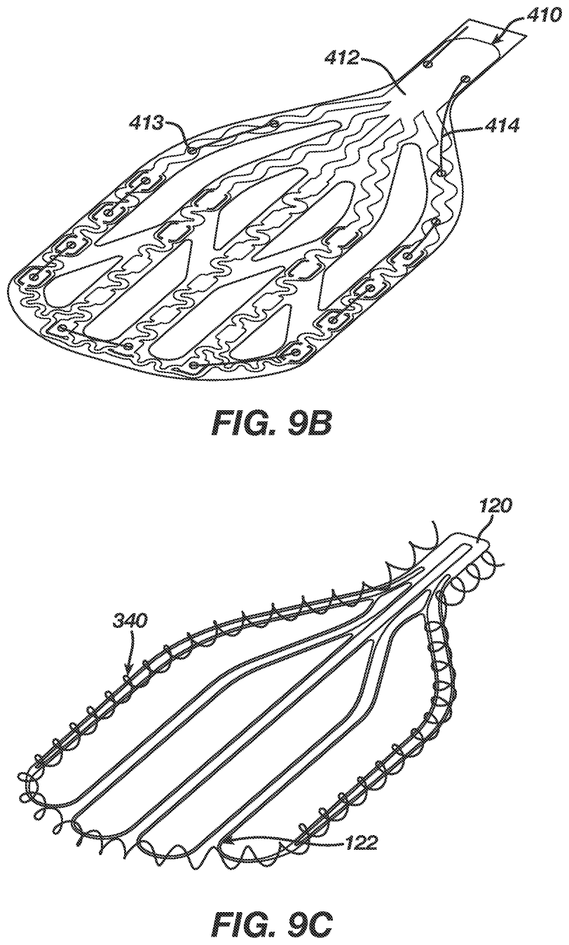

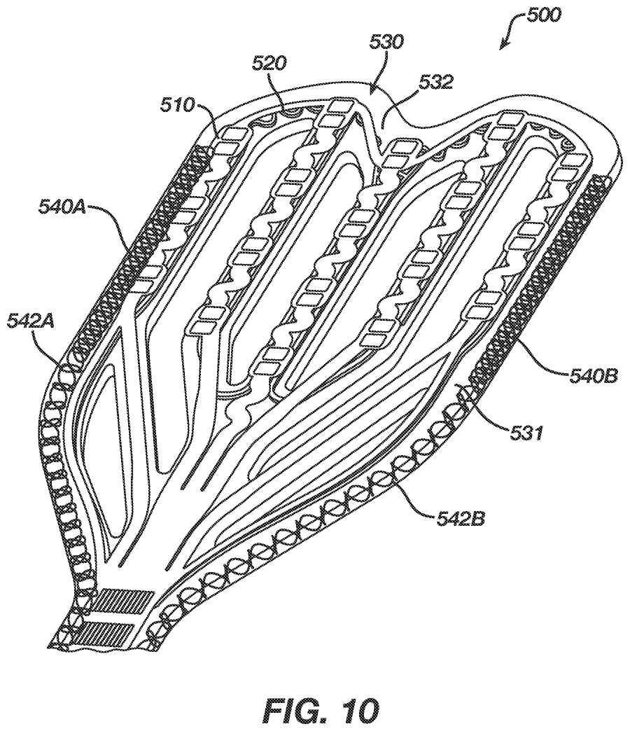

Alternatively or additionally, the substrate 112 can be stitched/sewn with the one or more conductive wires 114 can be sewn in one or more spiraling coils (similar to as depicted in FIG. 6A) and utilized as an electromagnetic sensing loop, further details of which are discussed below with respect to FIGS. 9A-10. The number of coils can be adjusted depending on the desired sensitivity of the sensor.

Stitching the conductive wires 114 to a medical grade fabric material 112A in the manner discussed above way provides many advantages over conventional flexible circuits. For example, it is cost-effective, scalable, and highly flexible in all direction. Moreover, a wire 114 with a circular cross-section, in contrast with a trace with a rectangular cross-section, does not have preferential bending/flexing directions and can bend in all directions which, in use, aids in the collapsibility and general reliability of the distal tip 100.

FIGS. 7A-7C show various views of another example of how the flexible circuit 110 of the present disclosure can be implemented. Rather than a fabric material 112A as the substrate 112, a bio-compatible thermoplastic material 112B (e.g., TPU) can be used as the flexible substrate 112. In this example, after the conductive wire(s) 114 is stitched/sewn to the thermoplastic material 112B, the thermoplastic material 112B can be laminated or reflowed onto other plastics, resulting in the conductive wire(s) 114 becoming suspended within a greater matrix of plastic, examples of which are discussed in greater detail with respect to FIGS. 9A and 10.

The conductive wire 114 can be routed along the flexible substrate 112B in a substantially straight direction along the longitudinal axis L-L with an alternating pattern between a first side of the thermoplastic material 112B and a second side of the thermoplastic material 112B, similar to what is shown in FIG. 7B (it is noted that the figures, e.g., FIGS. 7A-7C, show solid lines only for the purposes of illustration, and not to imply that the conductive wire does not or cannot pass from one side of the flexible substrate 112B to the other).

FIGS. 7B and 7C depict various a stitch pattern that can be employed with the thermoplastic material 112B. In particular, FIG. 7B (an unstretched configuration) depicts a zig-zag pattern that the conductive wire 114 is routed along the thermoplastic material 112B, which is formed from a stretchable material, in an alternating pattern between a first lateral direction (e.g., to the left relative to the longitudinal axis L-L) and a second lateral direction (e.g., to the right relative to the longitudinal axis L-L) relative to the longitudinal axis L-L. As shown in FIG. 7C (a stretched configuration), the conductive wire 114 can be stitched onto the stretchable substrate 112B such that the substrate can be stretched by, e.g., 30% along the longitudinal axis L-L without damage to the wire 114, even though the wire 114 itself is not meaningfully stretchable. This is possible because of the zig-zag pattern the wire 114 is initially stitched with (like the unstretched configuration of FIG. 7B), with the tightness of the zig-zag pattern changing depending on how much the stretchable material 112B is ultimately able to be stretched.

The examples depicted in FIGS. 7A-7C further include a thread 116 extending along the flexible substrate 112B, with the conductive wire 114 wrapping around the thread 116. The thread 116 aids in maintaining the stitch pattern of the conductive wire 114 when it is sewn/stitched to the thermoplastic material substrate 112B. In some examples, the thread 116 includes nylon. In other examples, the thread 116 can be formed from natural fibers (e.g., cotton), synthetic fibers other than nylons, thermoplastics (e.g., TPUs), or even other conductors or metals (e.g., Nitinol). In some examples, to make the depicted flexible circuit, a conductive wire 114 is sewn from a bobbin in a first direction, and the thread 116 is sewn from a spool in a second, opposite direction. Alternatively, conductive wire 114 can be sewn from both the bobbin and the spool, which enables integration of multiple traces at a time. Also, depending on the stitch pattern, wire 114 that is sewn from opposing directions can result in a twisted pair of wires 114 that can be used for electromagnetic noise cancellation. As exemplarily depicted in FIG. 7A, the conductive wire 114 and thread 116 can go in any direction and cross over one-another any number of times. Rather than requiring a new layer if lines are crossed (like in convention flexible circuit), in the present example, the wires 114 are sewn into the same substrate, and a new layer is created only in the location of the crossover.

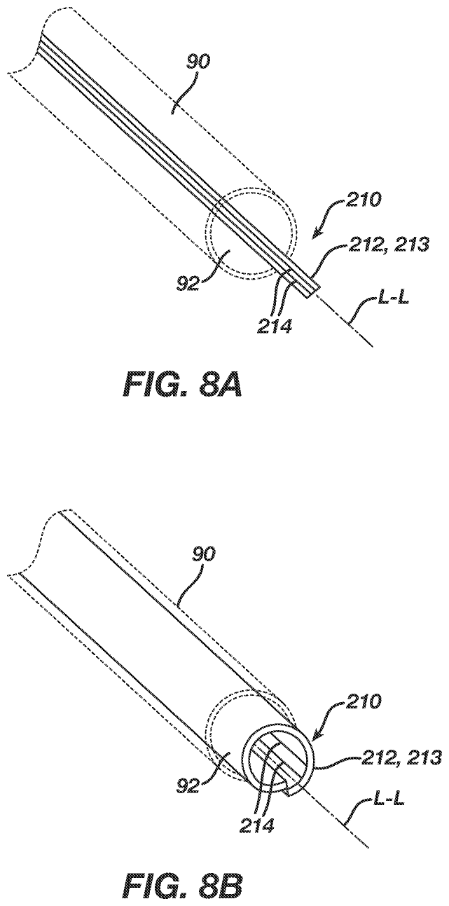

Turning now to FIGS. 8A and 8B, besides use in a distal tip 100 of a medical probe 14, the previously described flexible circuits 110 can be integrated with the elongated shaft 90 of the medical probe 14 that runs between the proximal and distal end thereof and connects with the probe tip 100. More specifically, the flexible circuit 110 can be configured as a long flexible circuit 210 that extends within the elongated shaft 90 therethrough. Like the previous examples, the long flexible circuit 210 includes a flexible substrate 212 and one or more conductive wires 214. Like previous examples, the flexible substrate 212 comprises a bio-compatible material (e.g., a fabric or thermoplastic as discussed above) and extending along a substrate plane 213 having a first side and a second side. Also similar to previous examples, the conductive wire 214 is routed along the flexible substrate 212 and connected thereto by a stitch pattern. The conductive wire 214 electrically connects to the probe tip in any appropriate manner, e.g., via a soldering pad at a base of the probe tip.

Due to the unique material properties of the flexible substrate 212, various cross-sectional profiles thereof can be employed, such as a rectangular cross-sectional profile (FIG. 8A) or a circular cross-sectional profile (FIG. 8B) where a wider piece of material is used for the flexible substrate 212 and it is rolled into a substantially cylinder shape before introducing it into the elongated shaft 90.

In another aspect of the present disclosure, FIG. 9A shows a perspective view of another probe tip 300. This example illustrates another manner in which the stitch pattern feature of the previously discussed examples can be applied in other ways to the probe tip 28 of the medical probe 14.

The probe tip 300 is configured for insertion into an internal body cavity of a patient and can include a flexible circuit 310 including a plurality of electrodes, a framework 320, an insulative material 330 in which the flexible circuit 310 and the framework are disposed, and a conductive wire 340. The flexible circuit can comprise one of the aforementioned materials (e.g., polyimide or a medical grade fabric) and can be generally configured in a similar manner as the previously described flexible circuit 110 or any other appropriate flexible circuit. In a similar vein, framework 320 can be configured equivalently as that of framework 120.

In the example of FIG. 9A, the insulative material 330 (or at least a portion thereof) is as functional as a flexible circuit and includes a flexible substrate 332 that includes a bio-compatible material (e.g., like the thermoplastic material used in FIGS. 7A-7C) and extends along a substrate plane 331 which bisects the substrate 332 and has a first side and a second side. The conductive wire 340 is routed along the flexible substrate 332 and connected to thereto by a stitch pattern (e.g., any of the stitch patterns previously mentioned, such as a straight stitch, zig-zag stitch, etc.). The conductive wire 340 functions as an electromagnetic sensing loop by forming a coil around a perimeter/periphery of the insulative material 330 that is configured to generate a current that is indicative of a position of the coil when the coil is subjected to a magnetic field.

The coil has a surface area (i.e., the area enclosed thereby). The amount of energy each sensing coil receives, when subjected to a magnetic field, is proportionate to the size of the coil. The conductive wire 340 can comprise electrical leads for conduction of current induced on the coil to the patient interface unit 30.

In some examples, the coil has multiple spiral loops 342, each spiraling loop 342 being constituted by a full revolution of the wire 340 in the helical pattern shown in FIG. 9A. In some examples each spiraling loop 342 includes a first section 342A disposed on the first side of the substrate plane 331 and a second section 342B disposed on the second side of the substrate plane 332. The spiraling loops 342 can be formed as a result of the wire 340 being sewn to the substrate 332. This pattern results in a design that prevents against potential delamination.

FIGS. 9B and 9C depict variants of the example of FIG. 9A. As seen in FIG. 9B, a flexible circuit 410 includes a substrate 412 that has a plurality of eyelets 413, with a conductive wire 414 sewn through the eyelets to form the sensing loop. While shown in the flexible circuit 410, it is also noted that these eyelets can be formed in the framework (e.g., framework 120). As seen in FIG. 9C, the conductive wire 340 can also be sewn/wrapped directly around the framework 120 with a plurality of spiraling loops (like the spiraling loops 342 in FIG. 9A) that extend around a portion of the framework 120. For example, the framework 120 can include one or more spines 122, with at least some of the spiral loops encircling portions of the spines 122 to retain it thereto.

In another aspect of the present disclosure, and similar to the example discussed with respect to FIG. 9A, FIG. 10 shows a perspective view of another probe tip 500. This example illustrates another manner in which the stitch pattern feature of the previously discussed examples can be applied in other ways to the probe tip 28 of the medical probe 14.

The probe tip 500 is configured for insertion into an internal body cavity of a patient and can include a flexible circuit 510 including a plurality of electrodes, a framework 520, an insulative material 530 in which the flexible circuit 510 and the framework 520 are disposed, a first conductive wire 540A, and a second conductive wire 540B. The flexible circuit 510 can comprise one of the aforementioned materials (e.g., polyimide or a medical grade fabric) and can be generally configured in a similar manner as the previously described flexible circuit 110 or any other appropriate flexible circuit. In a similar vein, framework 520 can be configured equivalently as that of framework 120.

In the example of FIG. 10, the insulative material 530 (or at least a portion thereof) is as functional as a flexible circuit and includes a flexible substrate 532 that includes a bio-compatible material (e.g., like the thermoplastic material used in FIGS. 7A-7C) and extends along a substrate plane 531 which bisects the substrate 532 and has a first side and a second side. The conductive wires 540A, 540B are routed along the flexible substrate 532 and connected to thereto by a stitch pattern. Compared with the example of FIG. 9A, which uses one conductive wire 340, the two conductive wires 540A, 540B are sewn/stitched down opposing lateral sides of the probe tip 500 to form a pair of electromagnetic sensor coils.

Each coil has multiple spiral loops 542A, 542B, each spiraling loop 542A, 542B being constituted by a full revolution of the wire 540A, 540B in the helical pattern shown in FIG. 10. In some examples each spiraling loop 542A, 542B includes a first section disposed on the first side of the substrate plane 531 and a second section 542B disposed on the second side of the substrate plane 532. The spiraling loops 542A, 542B can be formed as a result of the wires 540A, 540B being sewn to the substrate 532, which results in a design that prevents against potential delamination.

As seen, the pitch of the loops 542A, 542B is much tighter along sides of the probe tip 500 that run substantially parallel to the longitudinal axis L-L for use as a pair of sensing coils. As will be appreciated by those skilled in the art, the pitch can be loose enough to allow required flexibility. Deformation of the distal tip 500 will result in deformation of the respective loops 542A, 542B, and the resultant shift in sensitivity of the sensing coils can be used to calculate deformation of the distal tip 500. In other examples, the pitch on the loops 542A, 542B can be tight in localized locations, with the length thereof limited to enable location sensing while not compromising flexibility.

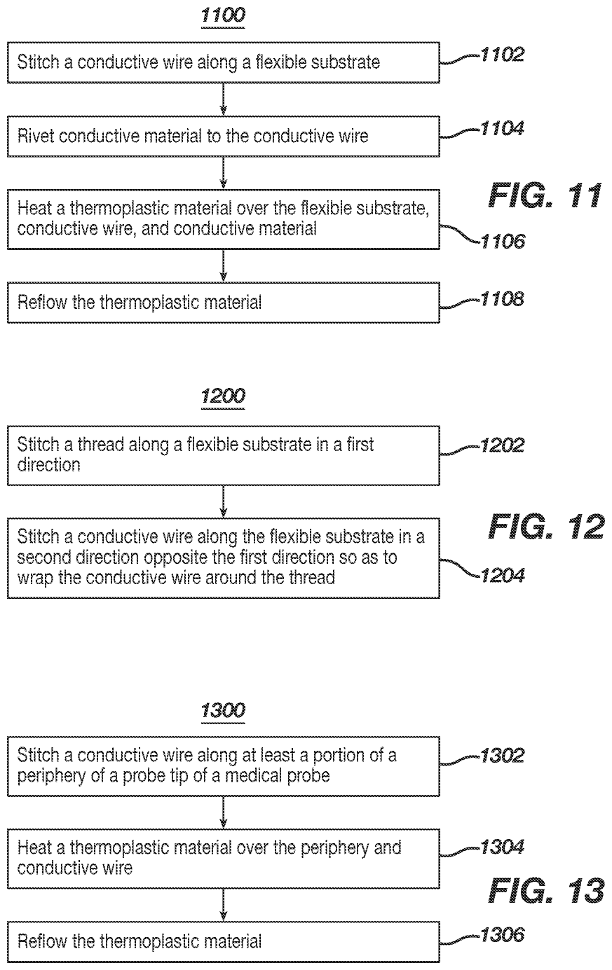

FIGS. 11-13 are flow charts that schematically illustrate various exemplary methods of making one or more portions of the presently described medical probe, in accordance with the present disclosure. Other implementations of the principles of these methods, such as the ones described above, are also considered to be within the scope of the present invention.

Making reference to FIG. 11, a method 1100 of forming a probe tip of a medical probe includes the following. A flexible circuit is formed by stitching 1102, using at least one of a sewn stitch, a woven stitch, or a patched stitch, a conductive wire along a flexible substrate (e.g., formed from a medical grade fabric material or a thermoplastic material). In instances where the flexible circuit is used for mapping or ablation, conductive material is riveted 1104 to the conductive wire to function as one or more electrodes. After the other layers of the medical probe have been positioned/stacked, a thermoplastic material is heated 1106 over the flexible substrate, conductive wire, and conductive material to reflow 1108 the thermoplastic material.

Turning now to FIG. 12, a method 1200 of stitching a conductive wire to a flexible substrate (e.g., a thermoplastic material, as depicted in FIGS. 7A-7C) includes the following. A thread is stitched 1202 along the flexible substrate. A conductive wire is stitched along the flexible substrate in a second direction, opposite the first direction, so as to wrap the conductive wire around the thread. It is noted that the stitching can be, e.g., in a straight line pattern, a zig-zag pattern, and/or in a loop/spiral pattern.

With reference to FIG. 13, another method 1300 of forming a probe tip of a medical probe includes the following. A conductive wire is stitched 1302 along at least a portion of a periphery of a probe tip a medical probe (e.g., as exemplified in FIGS. 9A and 10). The stitching comprises at least one of a sewn stitch, a woven stitch, or a patched stitch. After the other layers of the medical probe have been positioned/stacked, a thermoplastic material is heated 1106 over the flexible substrate and conductive wire, including the periphery of the probe tip, to reflow 1108 the thermoplastic material.

The disclosed technology described herein can be further understood according to the following clauses:

Clause 1. A flexible circuit configured for insertion into an internal body cavity of a patient, the flexible circuit comprising: a flexible substrate comprising a bio-compatible material and extending along a substrate plane; a plurality of electrodes disposed on the flexible substrate; and a conductive wire routed along the flexible substrate and connected to one or more of the plurality of electrodes by a stitch pattern.

Clause 2. The flexible circuit of clause 1, the stitch pattern comprising at least one of a sewn stitch, a woven stitch, a patched stitch, and combinations thereof.

Clause 3. The flexible circuit of any one of clauses 1-2, the conductive wire being routed along the flexible substrate in an alternating pattern between a first side of the substrate plane and a second side of the substrate plane.

Clause 4. The flexible circuit of any one of clauses 1-3, the flexible circuit extending along a longitudinal axis, and the conductive wire is routed along the flexible substrate in an alternating pattern between a first lateral direction and a second lateral direction relative to the longitudinal axis.

Clause 5. The flexible circuit of any one of clauses 1-4, the conductive wire being spirally routed along the flexible substrate.

Clause 6. The flexible circuit of any one of clauses 1-5, the bio-compatible material comprising a medical grade fabric material.

Clause 7. The flexible circuit of clause 6, the medical grade fabric material being conductive.

Clause 8. The flexible circuit of any one of clauses 1-5, the bio-compatible material comprising a thermoplastic material.

Clause 9. The flexible circuit of clause 8, the thermoplastic material comprising thermoplastic polyurethane.

Clause 10. The flexible circuit of any one of clauses 1-7, further comprising: a thread extending along the flexible substrate, with the conductive wire wrapping around the thread.

Clause 11. The flexible circuit of clause 10, the thread comprising nylon.

Clause 12. The flexible circuit of any one of clauses 1-11, the wire comprising copper, a Monel alloy, a nickel cobalt alloy with a silver core, Nitinol with a platinum core, Nitinol, or combinations thereof.

Clause 13. The flexible circuit of any one of clauses 1-10, the wire comprising twisted pairs of: magnet wire, copper and/or constantan wire, bundled shielded wires, or combinations thereof.

Clause 14. A medical probe configured for insertion into an internal body cavity of a patient and comprising: a flexible circuit comprising: a flexible substrate comprising a bio-compatible material and extending along a substrate plane having a first side and a second side; and a conductive wire routed along the flexible substrate and connected to the flexible substrate by a stitch pattern; and a plurality of electrodes electrically connected to the conductive wire on only the first side of the substrate plane.

Clause 15. The medical probe of clause 14, the conductive wire routed along the flexible substrate in an alternating pattern between the first side of the substrate plane and the second side of the substrate plane.

Clause 16. The medical probe of any one of clauses 14-15, the substrate plane bisecting the flexible substrate.

Clause 17. The medical probe of any one of clauses 14-16, further comprising a probe tip comprising: an insulative material; and a framework that is substantially planar along a longitudinal axis, the framework and the flexible circuit being disposed in the insulative material and spaced along a vertical axis.

Clause 18. A medical probe configured for insertion into an internal body cavity of a patient and comprising: an elongated shaft extending along a longitudinal axis; a probe tip connected to the elongated shaft; and a flexible circuit extending within the elongated shaft and comprising: a flexible substrate comprising a bio-compatible material and extending along a substrate plane having a first side and a second side; and a conductive wire routed along the flexible substrate and connected to the flexible substrate by a stitch pattern, the conductive wire being electrically connected to the probe tip.

Clause 19. The medical probe of clause 18, the flexible circuit forming a substantially cylindrical shape within the elongated shaft.

Clause 20. A medical probe configured for insertion into an internal body cavity of a patient and comprising: a flexible circuit comprising: a flexible substrate comprising a bio-compatible material and extending along a substrate plane having a first side and a second side; and a conductive wire routed along the flexible substrate and connected to the flexible substrate by a stitch pattern, the conductive wire forming a coil configured to generate a current that is indicative of a position of the coil when the coil is subjected to a magnetic field.

Clause 21. The medical probe of clause 20, the coil comprising a plurality of spiral loops, each loop comprising a first section disposed on the first side of the substrate plane and a second section disposed on the second side of the substrate plane.

Clause 22. The medical probe of any one of clauses 20-21, further comprising: a probe tip having a periphery, the coil extending along the periphery of the probe tip.

Clause 23. The medical probe of clause 20, further comprising: a probe tip comprising: the flexible circuit, a first lateral side, a second lateral side, and a distal end, the flexible circuit further comprising a second conductive wire, the conductive wire being spirally routed along the first lateral side, and the second conductive wire forming a second coil comprising a plurality of second spiral loops routed along the flexible substrate and the second lateral side.

Clause 24. The medical probe of any one of clauses 20-23, the flexible substrate comprising a plurality of eyelets, the conductive wire extending through each eyelet.

Clause 25. A medical probe configured for insertion into an internal body cavity of a patient and comprising: a framework that is substantially planar along a longitudinal axis; and a conductive wire that (i) forms a coil around a periphery of the framework and (ii) comprises a plurality of spiral loops, each loop extending around a portion of the framework.

Clause 26. The medical probe of clause 25, the framework comprising a plurality of spines, at least some of the spiral loops encircling respective portions of the spines.

Clause 27. The medical probe of clause 25, the framework comprising a plurality of eyelets, the conductive wire extending through the eyelets.

Clause 28. A method of forming a flexible circuit of a medical probe, the method comprising: stitching, using at least one of a sewn stitch, a woven stitch, or a patched stitch, a conductive wire along a flexible substrate.

Clause 29. The method of clause 28, the flexible substrate comprising a medical grade fabric material.

Clause 30. The method of clause 28, the flexible substrate comprising a thermoplastic material.

Clause 31. A method of forming a probe tip of a medical probe, the method comprising: stitching, using at least one of a sewn stitch, a woven stitch, or a patched stitch, a conductive wire along at least a portion of a periphery of the probe tip.

Clause 32. The method of clause 31, wherein stitching the conductive wire comprises sewing the conductive wire around the periphery of the probe tip.

Clause 33. The method of clause 31, wherein stitching the conductive wire comprises sewing the conductive wire along a first lateral side of the probe tip, and the method further comprises: stitching a second conductive wire along a second lateral side of the probe tip.

Clause 34. The method of any one of clauses 31-33, further comprising: heating a thermoplastic material over the periphery and the conductive wire; and reflowing the thermoplastic material.

The examples described above are cited by way of example, and the disclosed technology is not limited by what has been particularly shown and described hereinabove. Rather, the scope of the disclosed technology includes both combinations and sub combinations of the various features described hereinabove, as well as variations and modifications thereof which would occur to persons skilled in the art upon reading the foregoing description and which are not disclosed in the prior art.

What is claimed is:

1. A flexible circuit configured for insertion into an internal body cavity of a patient, the flexible circuit comprising:
a flexible substrate comprising a bio-compatible material and extending along a substrate plane;
a plurality of electrodes disposed on the flexible substrate; and
a conductive wire routed along the flexible substrate and connected to one or more electrodes of the plurality of electrodes by a stitch pattern.

2. The flexible circuit of claim 1, the stitch pattern comprising at least one of a sewn stitch, a woven stitch, a patched stitch, and combinations thereof.

3. The flexible circuit of claim 1, the conductive wire being routed along the flexible substrate in an alternating pattern between a first side of the substrate plane and a second side of the substrate plane.

4. The flexible circuit of claim 1, the flexible circuit extending along a longitudinal axis, and the conductive wire is routed along the flexible substrate in an alternating pattern between a first lateral direction and a second lateral direction relative to the longitudinal axis.

5. The flexible circuit of claim 1, the conductive wire being spirally routed along the flexible substrate.

6. The flexible circuit of claim 1, the bio-compatible material comprising a medical grade fabric material.

7. The flexible circuit of claim 6, the medical grade fabric material being conductive.

8. The flexible circuit of claim 1, the bio-compatible material comprising a thermoplastic material.

9. The flexible circuit of claim 1, further comprising:
a thread extending along the flexible substrate, with the conductive wire wrapping around the thread.

10. The flexible circuit of claim 1, the conductive wire comprising copper, a Monel alloy, a nickel cobalt alloy with a silver core, Nitinol with a platinum core, Nitinol, or combinations thereof.

11. The flexible circuit of claim 1, the conductive wire comprising twisted pairs of: magnet wire, copper and/or constantan wire, bundled shielded wires, or combinations thereof.

12. A medical probe configured for insertion into an internal body cavity of a patient and comprising:
a flexible circuit comprising:
a flexible substrate comprising a bio-compatible material and extending along a substrate plane having a first side and a second side; and
a conductive wire routed along the flexible substrate and connected to the flexible substrate by a stitch pattern; and
a plurality of electrodes electrically connected to the conductive wire on only the first side of the substrate plane.

13. The medical probe of claim 12, the conductive wire routed along the flexible substrate in an alternating pattern between the first side of the substrate plane and the second side of the substrate plane.

14. The medical probe of claim 12, the substrate plane bisecting the flexible substrate.

15. The medical probe of claim 12, further comprising a probe tip comprising:
an insulative material; and
a framework that is substantially planar along a longitudinal axis,
the framework and the flexible circuit being disposed in the insulative material and spaced along a vertical axis.

16. A medical probe configured for insertion into an internal body cavity of a patient and comprising:
a flexible circuit comprising:
a flexible substrate comprising a bio-compatible material and extending along a substrate plane having a first side and a second side; and
a conductive wire routed along the flexible substrate and connected to the flexible substrate by a stitch pattern, the conductive wire forming a coil configured to generate a current that is indicative of a position of the coil when the coil is subjected to a magnetic field.

17. The medical probe of claim 16, the coil comprising a plurality of spiral loops, each loop comprising a first section disposed on the first side of the substrate plane and a second section disposed on the second side of the substrate plane.

18. The medical probe of claim 16, further comprising:
a probe tip having a periphery, the coil extending along the periphery of the probe tip.

19. The medical probe of claim 16, further comprising:
a probe tip comprising: the flexible circuit, a first lateral side, a second lateral side, and a distal end,
the flexible circuit further comprising a second conductive wire,
the conductive wire being spirally routed along the first lateral side, and
the second conductive wire forming a second coil comprising a plurality of second spiral loops routed along the flexible substrate and the second lateral side.

20. The medical probe of claim 16, the flexible substrate comprising a plurality of eyelets, the conductive wire extending through each eyelet.
